(19) United States
(12) Patent Application Publication
(10) Pub. No.: US 2017/0202574 A1
(43) Pub. Date: Jul. 20, 2017

(54) Devices and Methods for Treating Occlusion of the Ophthalmic Artery

(71) Applicant: J.D. Franco & Company, Baltimore, MD (US)

(72) Inventors: Jeff Franco, Clarksville, MD (US); Michael Calhoun, Lighthouse Point, FL (US)

(21) Appl. No.: 14/385,496

(22) PCT Filed: Aug. 5, 2013
(86) PCT No.: PCT/US13/53670

§ 371 Date: Sep. 15, 2014

Related U.S. Application Data
(60) Provisional application No. 61/679,351, filed on Aug. 3, 2012.

(51) Int. Cl. A61B 17/3207; A61B 17/221; A61F 2/01; A61F 9/007

(52) U.S. Cl. CPC A61B 17/320725; A61F 9/00781; A61F 9/00736; A61B 17/221; A61F 2/013; A61F 2002/018

(57) ABSTRACT

There is provided herein a disclosure and specification of invention(s) relating to devices and methods for percutaneous access and treatment of vascular structures in the rear of the eye, including treatment for the symptoms related to Wet Age Related Macular Degeneration by removal of stenosis of the OA, thereby restoring normal, or near normal, blood flow to the rear of the eye, including the retina and associated structures. Also provided herein is a disclosure and specification of invention(s) relating to methods and devices for selective manipulation of Intraocular Pressure (IOP) be means of mechanical force for the purpose of inducing retrograde flow in the ophthalmic vasculature.

### FIELD OF THE INVENTION

The invention relates to medical devices and therapies for treating occlusion of the ophthalmic artery, and in particular to novel interventional devices to restoring and/or increasing vascular blood flow to the rear of the eye.

### BACKGROUND

Diseases of the eye, specifically Wet Age-related Macular Degeneration (WAMD), glaucoma, and diabetic retinopathy affect a large percentage of the population. However, current therapies are deficient in one or more aspects, necessitating improved approaches. The present inventive subject matter addresses some or all of the problems found in current therapies.

### SUMMARY OF THE INVENTION

There is provided herein a disclosure and specification of invention(s) relating to devices and methods for percutaneous access and treatment of vascular structures in the rear of the eye, including treatment for the symptoms related to Wet Age Related Macular Degeneration by removal of stenosis of the Ophthalmic Artery (OA), thereby restoring normal, or near normal, blood flow to the rear of the eye, including the retina and associated structures. Also provided herein is a disclosure and specification of invention(s) relating to methods and devices for selective manipulation of Intraocular Pressure (IOP) be means of mechanical force for the purpose of inducing retrograde flow in the ophthalmic vasculature.

In a preferred embodiment, there is provided an apparatus for treating obstruction of the ophthalmic artery, comprising an IOP device for mechanically applying a force against the front of the eye to increase Intraocular Pressure (IOP) sufficient to temporarily stop antegrade blood flow in the ophthalmic vasculature at the back of the eye and thereby induce retrograde flow in the ophthalmic vasculature; an atherectomy kit for performing an atherectomy upon the ophthalmic artery of a patient in need thereof; and a debris capture device for placement within the ophthalmic vasculature to capture artherectomy debris.

In another preferred embodiment, there is provided wherein the device uses mechanical force selected from the group consisting of hydraulic force, pneumatic force, gravitational force, spring force, and user-applied force, to contact the anterior portion of the eye(s) for the purpose of IOP manipulation, and wherein the apparatus is configured for use on one eye or on both eyes simultaneously.

In another preferred embodiment, there is provided wherein the apparatus uses mechanical force applied to the anterior portion either directly, or through the closed eyelid.

In another preferred embodiment, there is provided wherein the apparatus contains the capability to measure the IOP.

In another preferred embodiment, there is provided wherein the apparatus may measure the IOP using a sensor implanted within the vitreous cavity that is capable of assessing IOP values and transmitting data wirelessly.

In another preferred embodiment, there is provided wherein this wireless data transmission is provided in a continuous and real time manner.

In another preferred embodiment, there is provided wherein IOP is measured with a sensor temporarily placed within the vitreous cavity via a wired or wireless manner.

In another preferred embodiment, there is provided a feedback mechanism is provided for receiving IOP values (data) and provides for monitoring capability.

In another preferred embodiment, there is provided a feedback mechanism that provides for the general control of the IOP manipulation such that the IOP may be increased or decreased as deemed necessary.

In another preferred embodiment, there is provided a feedback mechanism is combined with a control function that allows for the ability to control the rate of increase and/or decrease of IOP as deemed necessary.

In another preferred embodiment, there is provided a feedback mechanism combined with a control function such the IOP values may be increased, decreased, maintained or cycled as necessary.

In another preferred embodiment, there is provided a feedback mechanism combined with a control function such the rate of IOP increase, decrease or steady state may be controlled.

In another preferred embodiment, there is provided a feedback mechanism combined with a control function such that specific parameters related to IOP values, rates of force and time at force may be specified and controlled.

In another preferred embodiment, there is provided a feedback mechanism combined with a control function such that when specific parameters are not met, the user is informed.

In another preferred embodiment, there is provided a feedback mechanism combined with a control function such that information related to the IOP value is displayed for the user to see.

In another preferred embodiment, there is provided a feedback mechanism combined with a control function such that the data may be displayed, manipulated and/or captured in a method for record keeping.

In another preferred embodiment, there is provided a method of treating obstruction of the ophthalmic artery, comprising the steps of: inducing retrograde flow in the ophthalmic vasculature by applying a mechanical force against the front of the eye to increase Intraocular Pressure (IOP) sufficient to temporarily stop antegrade blood flow in the ophthalmic vasculature at the back of the eye; performing an atherectomy upon the ophthalmic artery of a patient in need thereof during retrograde blood flow; and deploying a debris capture device within the ophthalmic vasculature to capture atherectomy debris, wherein the retrograde flow blocks the debris from the atherectomy from flowing downstream and causing an ischemic event.

In another preferred embodiment, there is provided wherein the retinal arteries flow in reverse for a predetermined timeframe.

In another preferred embodiment, there is provided wherein intravascular debris within the retinal artery flow in reverse for a predetermined amount of time.

In another preferred embodiment, there is provided wherein the ophthalmic arteries flow in reverse for a predetermined timeframe.

In another preferred embodiment, there is provided wherein intravascular debris within the ophthalmic artery flow in reverse for a predetermined amount of time.

In another preferred embodiment, there is provided use in conjunction with an interventional device placed within the target anatomy for the purpose of tissue removal i.e; stenois, lesions, etc.

In another preferred embodiment, there is provided wherein debris is captured by placement of a capture device placed within the target anatomy.

In another preferred embodiment, there is provided a tissue removal device for treating obstruction of the ophthalmic artery, comprising: a percutaneously delivered tapered corewire ranging in diameter from about 0.19 mm to about 0.88 mm, the corewire disposed within a delivery sheath, said corewire having a tissue cutting element at or near a distal end, said corewire having an integral inflatable balloon section at the distal end as a protective element, and said corewire having an atraumatic tip.

In another preferred embodiment, there is provided wherein wherein the device is configured for percutaneous access of the Internal Carotid Artery (ICA).

In another preferred embodiment, there is provided wherein the device is configured for percutaneous access of the Ophthalmic Artery (OA).

In another preferred embodiment, there is provided wherein the device is configured to be visible using non-invasive imaging techniques (i.e: fluoroscopy).

In another preferred embodiment, there is provided wherein the device includes distal emboli protection.

In another preferred embodiment, there is provided a flow direction device to aid in the positioning of the device within the target anatomy.

In another preferred embodiment, there is provided a flow direction device that uses reverse flow to aid in the removal of the device from within the target anatomy during selectively induced retrograde flow.

In another preferred embodiment, there is provided a specifically shaped guidewire to access the OA from the ICA.

In another preferred embodiment, there is provided a specifically designed guiding catheter to access the OA from the ICA.

In another preferred embodiment, there is provided a specifically shaped guidewire to access the OA from the ICA, through the guiding catheter, once the guiding catheter has transited the OA, wherein this guidewire is configured to gain further downstream OA access without disturbance of vessel physiology due to guidewire tip shape.

In another preferred embodiment, there is provided a downstream protection element for downstream protection in the ICA.

In another preferred embodiment, there is provided a method of use of a shaped tip guidewire, a straight tip guidewire and a guiding catheter, comprising the steps in which the straight tip guidewire is used alone, or in conjunction with the guiding catheter to access the OA from the ICA, once the OA has been cannulated, the shaped tip guidewire is exchanged for the straight tip guidewire for the balance of the procedure.

In another preferred embodiment, there is provided a method of use as in claim 36 in which once the OA has been cannulated, the shaped tip guidewire is exchanged with an interventional device for the balance of the procedure.

In another preferred embodiment, there is provided an apparatus for capturing atherectomy debris as it is removed, comprising a single hypo tube cut to contain a combination atherectomy device and distal protection device.

In another preferred embodiment, there is provided wherein the atherectomy device portion fits within a delivery sheath such that the fully expanded diameter is achieved when device is moved out of sheath and into the target anatomy with that fully expanded diameter at 1.4 mm, which compliance to a vessel as small as 0.7 mm.

In another preferred embodiment, there is provided wherein the atherectomy device portion fits within a delivery sheath and the fully expanded diameter is achieved when device is moved out of sheath, into the target anatomy and a central, slideable corewire is manipulated to achieve the final diameter.

In another preferred embodiment, there is provided wherein the apparatus is constructed of a solid corewire with a mounted atherectomy and distal protection device.

In another preferred embodiment, there is provided wherein the solid corewire contains external geometry specific to the function of performing atherectomy work.

In another preferred embodiment, there is provided wherein the atherectomy portion of the apparatus is expandable.

In another preferred embodiment, there is provided wherein the atherectomy portion of the apparatus in non-expandable.

In another preferred embodiment, there is provided wherein the atherectomy portion of the apparatus is non-expandable, but rotatable such that rotation induces a diametric increase in the apparatus.

In another preferred embodiment, there is provided wherein the atherectomy device fits within a delivery sheath such that the fully expanded diameter is achieved when the device is moved out of sheath and into the target anatomy.

In another preferred embodiment, there is provided wherein the atherectomy device portion fits within a delivery sheath such that the non expanded diameter is revealed when the device is moved out of sheath and into the target anatomy.

In another preferred embodiment, there is provided wherein the apparatus is constructed of a balloon designed to inflate such that contact with the target anatomy is achieved.

In another preferred embodiment, there is provided wherein the balloon has external materials affixed directly to the balloon surface to facilitate atherectomy.

In another preferred embodiment, there is provided wherein the balloon has external emboli protection.

In another preferred embodiment, there is provided wherein the balloon is a balloon mounted on a polymer catheter typical of current vascular procedure technology.

In another preferred embodiment, there is provided wherein the balloon is a balloon mounted on a solid corewire.

In another preferred embodiment, there is provided wherein the balloon is mounted on a hypotube.

In another preferred embodiment, there is provided a device for the removal of debris by aspiration.

In another preferred embodiment, there is provided wherein the apparatus has a deployed, fully expanded diameter of 1.2 to 1.4 mm, compressible yet effective at 0.7 mm of deployed diameter.

In another preferred embodiment, there is provided wherein the apparatus has a deployed, fully expanded diameter of 1.0 to 1.6 mm, compressible yet effective at 0.7 mm of deployed diameter.

In another preferred embodiment, there is provided wherein the apparatus has a balloon shape optimized to affect removal of material.

In another preferred embodiment, there is provided wherein the apparatus has an aspiration device for removal of debris by aspiration via an external sheath.

In another preferred embodiment, there is provided wherein the apparatus is made of materials selected from nitinol, stainless steel, or other materials commonly associated with intravascular medical devices.

In another preferred embodiment, there is provided a method in which the apparatus is percutaneously inserted via the ICA and navigated to the OA.

In another preferred embodiment, there is provided wherein the navigation of the apparatus is guided by use of a non-invasive imaging methodology (ie: fluoroscopy).

In another preferred embodiment, there is provided the use of distal protection is provided for the ICA.

In another preferred embodiment, there is provided the use of distal protection is provided for the OA.

In another preferred embodiment, there is provided a method in which removal of debris by aspiration is provided for while in the OA.

In another preferred embodiment, there is provided a method for providing treatment for the symptoms related to Wet Age Related Macular Degeneration, comprising the step of removal of stenosis of the OA, thereby restoring normal, or near normal, blood flow to the rear of the eye, including the retina and associated structures.

In another preferred embodiment, there is provided a method for providing a pharmaceutical based treatment for the symptoms of Wet Age Related Macular Degeneration by delivery of a pharmaceutical compound(s) specifically targeted for the treatment of WAMD.

In another preferred embodiment, there is provided a method for providing a pharmaceutical treatment for the medication and/or restenosis of a specific section of the Ophthalmic Artery by delivery of a pharmaceutical compound(s) specifically targeted for the treatment of vascular lesions.

In another preferred embodiment, there is provided a method for providing a pharmaceutical treatment for the prevention and/or treatment of thrombus or thrombus related conditions in a specific section of the Ophthalmic Artery by delivery of a pharmaceutical compound(s) specifically targeted for the treatment of thrombus or thrombus related conditions.

In another preferred embodiment, there is provided an apparatus for providing a pharmaceutical based treatment for the symptoms of Wet Age Related Macular Degeneration by physical delivery of a pharmaceutical compound(s) specifically targeted for the treatment of WAMD.

In another preferred embodiment, there is provided an apparatus for providing a pharmaceutical treatment for the medication and/or restenosis of a specific section of the Ophthalmic Artery by physical delivery of a pharmaceutical compound(s) specifically targeted for the treatment of vascular lesions.

In another preferred embodiment, there is provided an apparatus for providing a pharmaceutical treatment for the prevention and/or treatment of thrombus or thrombus related conditions in a specific section of the Ophthalmic Artery by physical delivery of a pharmaceutical compound(s) specifically targeted for the treatment of thrombus or thrombus related conditions.

In another preferred embodiment, there is provided an apparatus packaged within a single unit, containing a hybrid catheter and flow directed balloon.

In another preferred embodiment, there is provided an apparatus where the OD of single unit being 6-9 french at the thicker, proximal end of the hybrid catheter/balloon with the distal 2-5 CM of the apparatus being 0.12-0.19 mm in diameter of a flow directed guidewire with the final distal portion of the apparatus being 8 to 15 mm of the apparatus being a flow directed balloon.

In another preferred embodiment, there is provided an apparatus where the flow directed balloon inflates to a maximum of 1.4 mm and a minimum of 0.7 mm.

In another preferred embodiment, there is provided an apparatus that coats the balloon with a drug for delivery and compression into the wall of the arterial source with the stenotic lesion.

### BRIEF DESCRIPTION OF FIGURES

FIGS. 1A and 1B are each a semi-transparent perspective side view of an embodiment of the present inventive subject matter.

FIG. 2 is a semi-transparent perspective side view of another embodiment of the present inventive subject matter.

FIG. 3A is a photo showing a corewire. FIG. 3B is a photo showing a tapered corewire.

FIG. 4 is side view of another embodiment of the present inventive subject matter.

FIGS. 5A and 5B are before and after side views of another embodiment of the present inventive subject matter.

FIG. 5C is a side view of another embodiment of the present inventive subject matter.

FIGS. 6A and 6B are a before and after side views of another embodiment of the present inventive subject matter.

FIG. 7 is a series of three line drawings showing a hypotube atherectomy corewire and expanded atherectomy balloon with distal protect element.

FIG. 8 is a two-part side view line drawing of a multicomponent apparatus of the present invention.

FIGS. 9A and 9B are before and after side views of another embodiment of the present inventive subject matter.

FIG. 10 is a series of three line drawings showing variations in balloon distal elements.

FIG. 11 is a series of three sequential line drawings showing use of shaped and straight guide wires.

FIG. 12 is a side view line drawing of an embodiment having an inflatable balloon and a intravascular positioning device/parachute.

FIG. 13 is a side view line drawing of an embodiment having an inflatable balloon and a intravascular positioning device/parachute.

FIG. 14 is a variation of a side view line drawing of an embodiment having an inflatable balloon and a intravascular positioning device/parachute.

FIG. 15 is a variation of a side view line drawing of an embodiment having an inflatable balloon.

FIG. 16 is a variation of a side view line drawing of an embodiment having an inflatable balloon.

FIG. 17 is chart showing anatomy and use of an IOP device.

FIG. 18 is a side view line drawing of the eye showing IOP caused by mechanical force.

FIG. 19 is a front view line drawing of the eye showing IOP caused by mechanical force, with a controller unit for interacting in a continuous or periodic manner.

FIG. 20 is an anatomical drawing of the eye for reference purposes only.

### DETAILED DESCRIPTION OF THE INVENTION

Without being limited to any specific theory, the invention is based on the premise that the primary causative effect for Wet Age-Related Macular Degeneration (WAMD), glaucoma and diabetic retinopathy is occlusion of the Ophthalmic Artery (OA) such that normal blood flow is restricted (ischemia) to the rear of the eye. As a result of this ischemia, hypoxia (resulting in neovascularization) is induced in these structures and vision eventually devolves into a dysfunctional retina (WAMD). From this, we have identified two designs that may be used to provide a treatment methodology for WAMD. Several variations are detailed later in this specification. These include, 1) a device(s) for performing interventional work in the ophthalmic artery and surrounding structures to restore/increase vascular blood flow and, 2) a device for selectively inducing retrograde blood flow in the retinal vasculature via manipulation for intraocular pressure (IOP).

Interventional Device

The interventional device is designed to gain access to and deliver direct mechanical and/or drug therapy to a specific location of the anatomy. While the following examples specifically detail the necessary components for a particular ophthalmic artery (OA) application, this technology may be used in any anatomical location in which removal of material is desired in a luminal environment. This environment may be vascular or not and may be used in any tubal, luminal or other similar anatomical structure where removal of material is desired. As such, the invention can be scaled, modified or constructed such that it can provide therapy for a specific luminal anatomical location/need. The general inventive device design is based on a central wire, hypotube, coil, balloon or combination thereof. The inventive device is made of stainless, nitinol, polymer, other materials or a combination thereof and designed to accommodate specific approaches (carotid, subclavian, femoral, endoscopic or laparoscopic). For the example given, entrance into the body is provided by a vascular access element which may be typical, or may be designed specifically for use with the inventive device (ie: catheter sheath introducer or equivalent). The inventive device fits within a sheath, which is designed to provide a protective element for the device as well as to prevent vessel trauma during delivery to the target site. The distal portion includes the ability to provide distal protection in the OA, as well as an element to provide diametric interference. This area of diametric interference is designed to interface with the target vessel segment (eg; lesion) such that specific and deliberate manipulation provides for the ability to selectively remove material from the lesion site. The diametric interference element also provides for the ability to compress such that it fits within the device sheath to provide a minimal diametric dimension. This diametric portion is also referred to as an interventional element. Once the device is placed at the target anatomy, the interventional element is positioned such that it is outside the sheath and it conformally fits the inner diameter of the target anatomy. The interventional element also contains a design element that allows for tissue removal when manipulated in a specific manner. That manner includes manual rotation, manual push/pull, mechanical rotation, mechanical push/pull, site specific drug delivery or a combination of some or all of those. Additionally, the tissue removal device and conforming element is optionally different devices, two devices or different segments of the same device. Once material removal is complete, the interventional element is pulled into the sheath, along with the distal protection portion (equipped) of the device and the entire assembly removed. It is also possible to remove the interventional element for cleaning and to replace and continue. Furthermore, this device is able to deliver drug therapy directly to the area of intervention. For example, delivery of a pharmaceutical compound to reduce the rate of restenosis may be possible as well as a variety of other pharmaceutical compounds. The device is also constructed such that it is able to provide interventional therapy in the form of energy delivery. This includes, but is not limited to, laser, ultrasound, cryogenic, radiofrequency (RF) and/or other energies or combination thereof. Additionally, there is also the provision for the ability to provide direct optical viewing of the target site prior to, during and after administration of therapy. There is an ability to combine multiple drug therapies for a single condition or multiple conditions. For example, Sirolimus for antiproliferative effect post angioplasty. In addition to this or separate from this, a statin may be included and eluted as lipid like deposits called drusen can be concomitant to Wet AMD. It is presumed that the slow elution of a statin would reduce the size and number of drusen deposits and there by improve eyesight.

Interventional Device—Common Device Elements:
1. Ability to visualize under fluoroscopy
2. Preferred internal carotid access (can be done via subclavian or femoral)
3. Distal protection element in the internal carotid artery (ICA)
4. Distal protection element in the Ophthalmic Artery
5. Works in OA diameter ranges between 0.7 to 1.4 mm—derived by atmospheric pressure applied to the conformal element
6. Working length for OA estimated to be about 15 inches, further definitions included
7. Approaches other than ICA also included
8. Ability to remove material from the OA and transport out of the vasculature
9. Ability to induce retrograde flow, either continuously, or on demand for specific time periods.
10. May use of a guiding catheter to cannulate the OA from the ICA (combination of GC features with sheath to have an ‘all in one’)

Interventional Device—Singular Elements (Specific to a Particular Design):
1. Distal OA protection as an integral element of the device
2. Distal OA protection as a separately placed/removed device
3. Distal CA protection as an integral element of the device
4. Distal CA protection as a separately placed/removed device
5. Distal ICA protection as a integral placed/removed device
6. Distal ICA protection as a separately placed/removed device.
7. Ability to deliver an RF element for therapy
8. Ability to deliver a laser element for therapy
9. Ability to deliver an ultrasound element for therapy
10. Ability to deliver a cryogenic element for therapy
11. Ability to deliver drugs via infusion
12. Ability to deliver drugs via injection (bolus—TPA)
13. Drug delivery capability before, during and after material removal
14. Ability to deliver drugs via micro needles

IA. Interventional Device—Specific Examples: Solid Core Wire Based

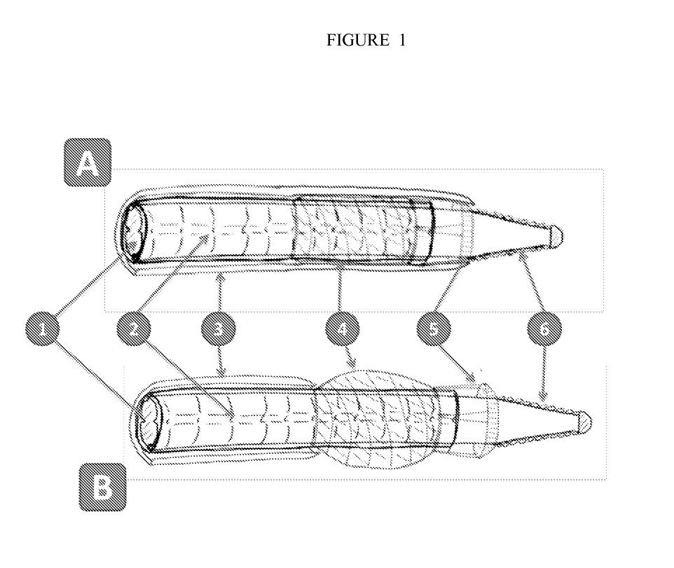

FIG. 1 shows an embodiment of the present invention having an Aspiration Core. The design is based on a solid metallic corewire with integrated aspiration capability. The device consists of the following elements and features as detailed in FIG. 1:
1. Center corewire
2. Longitudinal indentations
3. Delivery sheath
4. Cutting element
5. Distal protection element
6. Atraumatic tip

FIG. 1A depicts the device with the delivery sheath (3) covering the cutting (4) and distal protection (5) elements, which are both mounted on the central core (1). The tip of the device contains an atraumatic tip (6) to aid in placement of the device.

FIG. 1B (above) depicts the delivery sheath pulled back and the cutting and distal protection elements both in a deployed position. Aspiration is accomplished by either flushing and aspirating using alternate longitudinal channels (2) of the corewire, or by a combination use of longitudinal channels and the delivery sheath, one for flushing and the other for aspiration. Once the procedure is complete, the device is withdrawn back into the delivery sheath and positioned as seen in FIG. 1A. The device is then safely withdrawn from the anatomy.

Generally, the overall length of the device is optimized for the anatomical location and approach. In a preferred example, for use within the OA, an overall length of about 160 cm or about 15.00 inches for the device would be used in conjunction with an appropriately designed sheath. The maximum overall diameter of the sheath would be in the 1.0 mm range (after inflation), with the cutting and distal protection elements offering a conformal fit capability in the deployed range of between 0.7 mm to 1.4 mm as dictated by the specific dimensions of the OA and the lesion site. Of course, these overall length and diametric dimensions would be adjusted based on the specific applications and is contemplated as within the scope of the invention. In addition, the specific material composition, formulation and manufacturing parameters of material used would be refined to address the specific application and is contemplated as within the scope of the invention. This dimensional information applies to all of the designs disclosed. In one preferred example, the lesion crossing profile of this device is less than 0.2 mm. A range of appropriate profile dimensions is contemplated as within the scope of the invention.

A. Interventional Device—Specific Examples: Plain Core—Non Aspiration Core

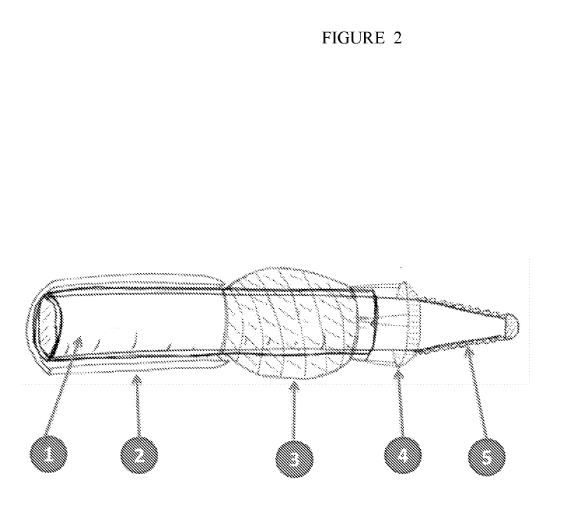

The design in FIG. 2 is based on a solid corewire and does not have specific aspiration capability. The device consists of the following elements and features:
1. Center corewire
2. Delivery sheath
3. Cutting element
4. Distal protection element
5. Atraumatic tip

This inventive subject matter of FIG. 2 is essentially the same as the FIG. 1 aspiration core with the exception that the core is note designed to facilitate aspiration. The remaining elements of the device are essentially similar to the aspiration core design. The drawing (above) depicts the delivery sheath pulled back and the cutting and distal protection elements both in a deployed position. Once the procedure is complete, the device is withdrawn back into the delivery sheath and positioned in a similar fashion as seen in the aspiration core FIG. 1A. The device is then safely withdrawn from the anatomy.

It should also be noted that the corewire based design may include elements that are much simpler in design than illustrated in the sketch above. These designs could include a wire with a specific drawn profile that is inserted into the anatomy such that movement of the wire would allow an interface between the profile of the corewire and the anatomy to facilitate lesion material removal. These particular designs could include 1) a straight ‘as drawn’ wire, 2) an as drawn wire with a twist or 3) a selective combination of the two.

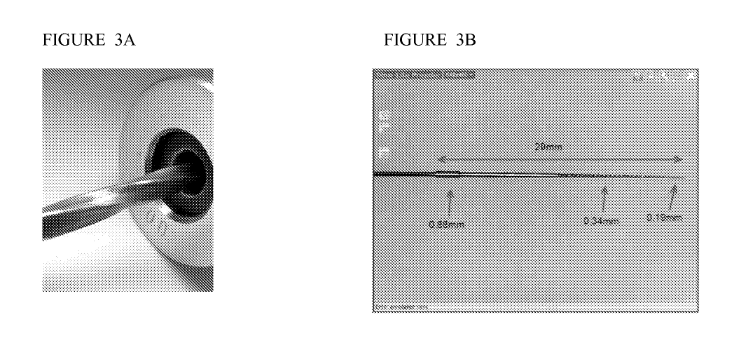

FIG. 3A and FIG. 3B depict drawn wire with a twist and the distal tip segment of our initial corewire based prototype design.

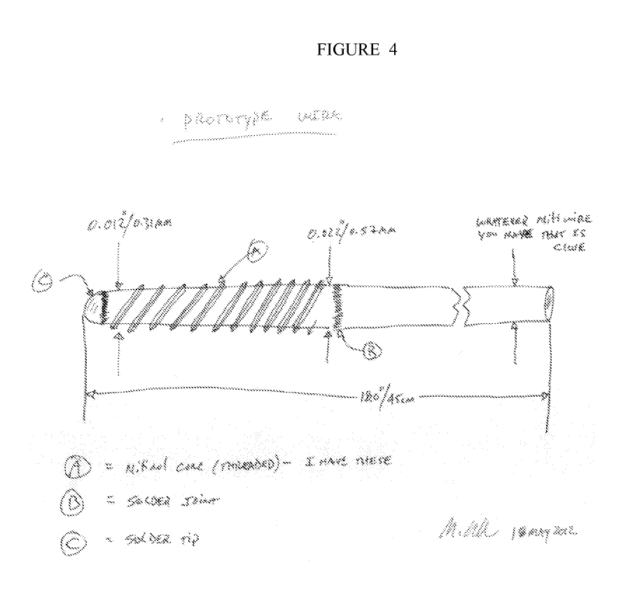

FIG. 4 depicts the initial corewire based prototype overall configuration.

B. Interventional Device—Hypotube Based

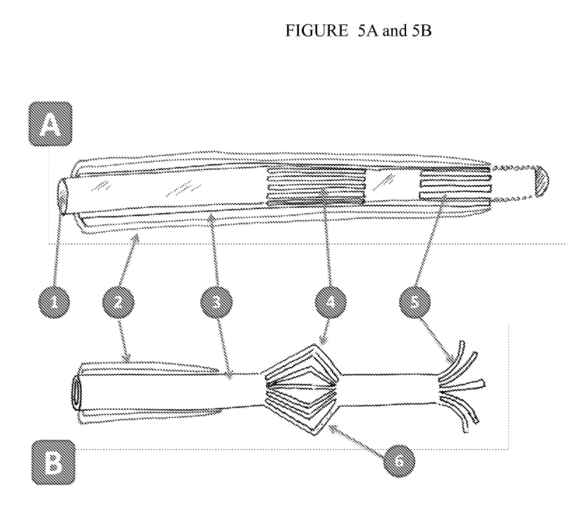

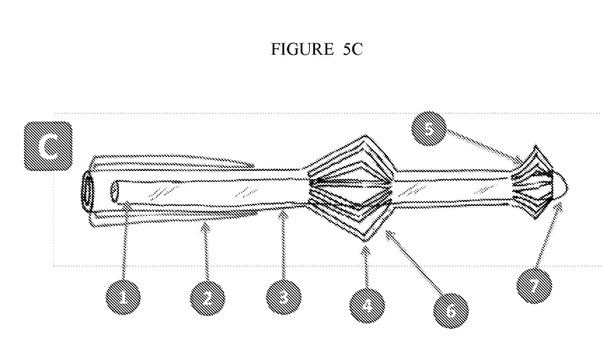

Integral elements—The design in FIG. 5 is based on a hollow metallic tube. Aspiration capability is not detailed in this sketch, but may be possible with the addition of a central flush source. The device consists of the following elements and features as detailed in FIG. 5A and FIG. 5B:

| | | |
|---|---|---|
| 1. Central guidewire | 2. Delivery sheath | 3. Hollow tube |
| 4. Cutting element | 5. Distal protection element | 6. Abrasives |

FIG. 5A depicts the device with the delivery sheath (2) covering the cutting (4) and distal protection (5) elements, which are both cut from the actual hypotube (3) and as such, are integral to the hypotube

An alternative version of this design would be a hypotube version with cutting and distal protection elements mounted on the hypotube. There would also be a provision for an element that would be positioned in the lumen after removal of the guidewire. This element would serve to deliver fluid for flushing. In this example, aspiration could be accomplished by applying suction to the proximal hypotube such that fluid is removed as well as debris while flushing is activated. While these inventions are not sketched, this document discloses such configuration). A guidewire (1) extends down the inner lumen of the hypotube to provide a means for navigating the anatomy. Upon placement within the target anatomy, the guidewire is removed and the sheath is pulled back, deploying the cutting and distal protection elements. Deployment of the distal elements is controlled by selective manufacturing processes which preferentially ‘train’ the elements to behave in a certain fashion such that they exhibit a condition known as ‘shape memory”. This shape memory is exhibited by the hypotube when it is in an unrestrained position. Abrasives (6) mounted, coated or integral with the cutting element may be designed to facilitate material removal and shaping of the lesion.

FIG. 5B depicts the delivery sheath pulled back and the cutting and distal protection elements both in a deployed position. Once the procedure is complete, the device is withdrawn back into the delivery sheath and positioned as seen in FIG. 5A. The device can then be safely withdrawn from the anatomy.

FIG. 5C depicts an alternative embodiment of the hypotube design. In this example, all elements are similar as in the previous sketch, with the exception of number 7. Element number 7 details a moveable internal core wire, which is joined with inner distal tip of the hypotube such that longitudinal movement of the corewire may serve to either expand elements 4 and 5, or compress them. When the procedure is complete, removal of this device would be accomplished in a similar fashion as describe in Drawing B, above.

IC. Interventional Device—Polymer Based Tube

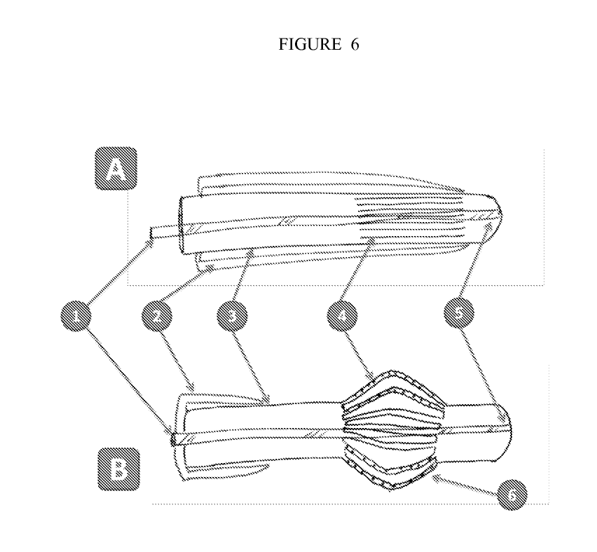

FIG. 6A depicts the device with the delivery sheath (2) covering the cutting element (4), which is cut from the polymer tube (3) and as such, are integral to the tube (Note: an alternative version of this design would include a provision for an element that would be positioned in the lumen after removal of the guidewire. This element would serve to deliver fluid for flushing. In this example, aspiration could be accomplished by applying suction to the proximal sheath such that fluid is removed as well as debris while flushing is activated. While this invention is not sketched, this document discloses such configuration).

FIG. 6B details a moveable internal corewire (1) which extends down the inner lumen of the tube and is fastened to the distal tip of the device (5) to provide a means for deploying the cutting and distal protection elements through either expansion or contraction. Abrasives (6) mounted, coated or integral with the cutting element may be designed to facilitate material removal and shaping of the lesion.

ID. Interventional Device

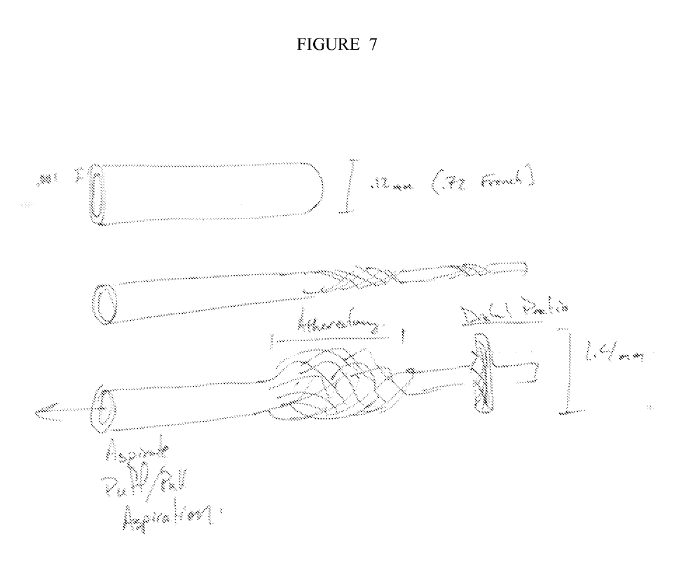

FIG. 7 shows a Single Hypotube based design in which a “puff/pull” aspiration of the atherectomy debris is applied. A single hypotube of 0.12 (0.10-0.14) mm with a 0.001″ thickness is laser cut and set to expand an atherectomy device and distal protection device. The device is used by puffing saline or another inert liquid into the space while simultaneous (manually or mechanically) aspirating the disease area and applying rotational force (pushing or turning, mechanically or manually) on the lesion. When fully deployed, the device is 1.4 millimeter in maximum diameter.

E. Interventional Device

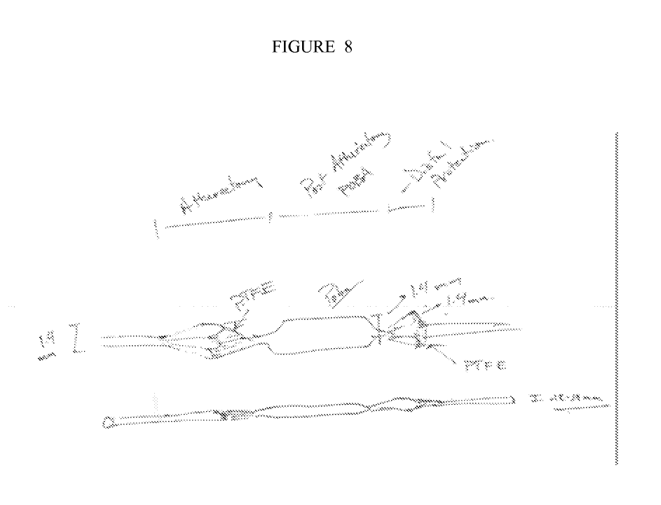

FIG. 8 shows a basket like atherectomy device, proximal to a POBA/DE Balloon, proximal to a distal protection device. The basket like atherectomy device and distal protection are deployed distal to the lesion. The device is pulled into the catheter, scrapping debris into the basket. As the balloon passes the lesion site after atherectomy an angioplasty is applied, facilitating a smooth, non-striated blood interface.

IF. Interventional Device—Balloon Based

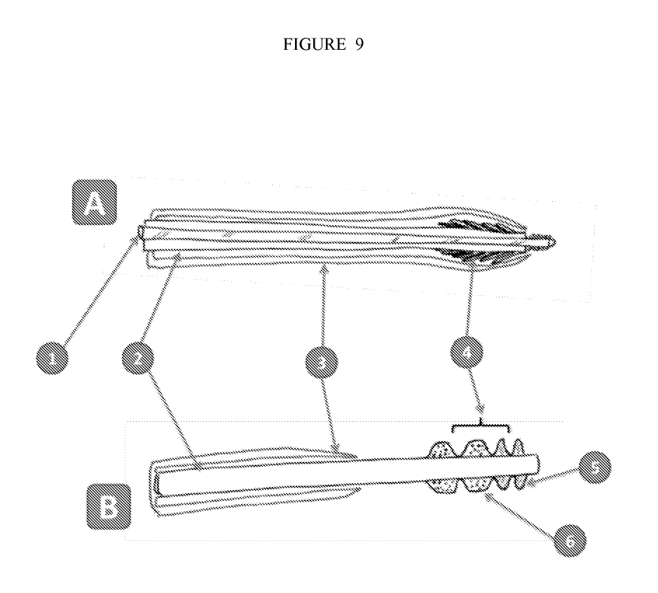

FIG. 9A depicts the device with the delivery sheath (3) covering the balloon catheter body (2) as well as the cutting (6) and distal protection (5) elements, which are both integral with the balloon body (4). (Note: There would also be a provision for an element that would be positioned in the lumen after removal of the guidewire. This element would serve to deliver fluid for flushing. In this example, aspiration could be accomplished by applying suction to the proximal sheath such that fluid is removed as well as debris while flushing is activated. While this invention is not sketched, this document discloses such configuration). A guidewire (1) extends down the inner lumen of the device to provide a means for navigating the anatomy.

FIG. 9B depicts placement within the target anatomy, where the guidewire is removed and the sheath pulled back, exposing the cutting and distal protection elements. Deployment of the distal elements is controlled by use of an inflation device to fill the balloon with fluid. Once the balloon is inflated, the profile would take shape such that the cutting and distal protection elements are deployed. Abrasives (6) mounted, coated or integral with the cutting element may be designed to facilitate material removal and shaping of the lesion. Hydrogels or other material (5) may be integral to the distal protection element such that material is attracted and adheres to it.

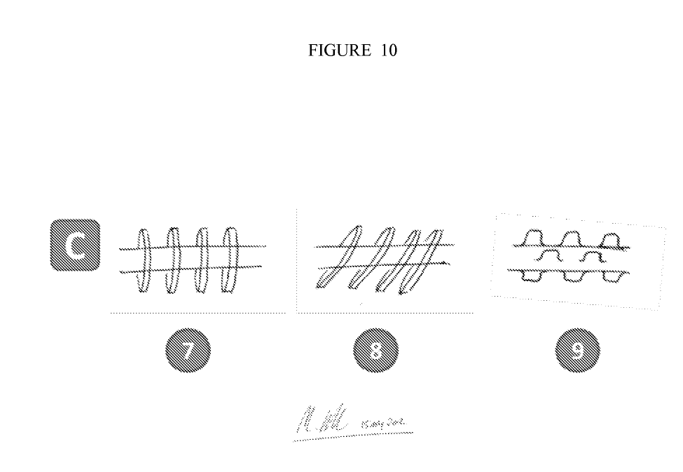

FIG. 10 depicts some general shapes for the balloon distal elements. These sketches serve to provide only general variation ideas and are not meant to be all inclusive.

IG. Interventional Device—Ophthalmic Artery Access Element

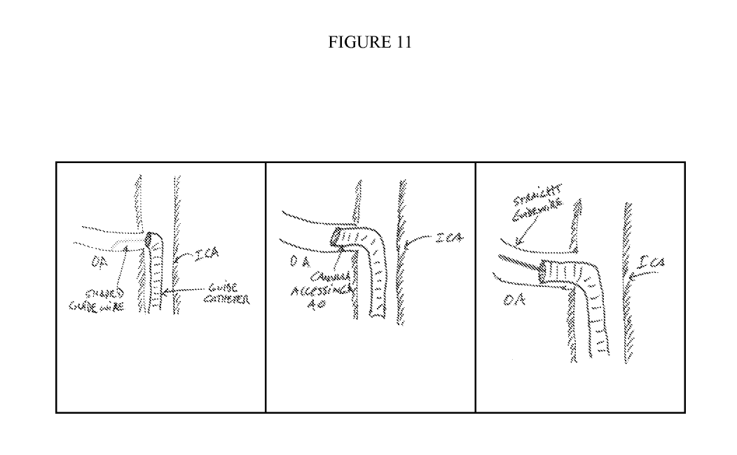

FIG. 11 shows the use of a shaped guidewire to access the OA and follow up with a guiding catheter to position within the entry to the OA. Once inside, the shaped guidewire will be exchanged for either a straight guidewire or an interventional device to continue the procedure. FIG. 11 depicts access of the OA by use of a shaped guidewire, entry into the OA by a guiding catheter over the shaped guidewire and finally exchange of the shaped guidewire for either a straight tip guidewire or an interventional device. The guidewire and guiding catheter is specifically designed for use in the OA and may include a provision for providing downstream protection.

IH. Interventional Device—Flow Directed 1

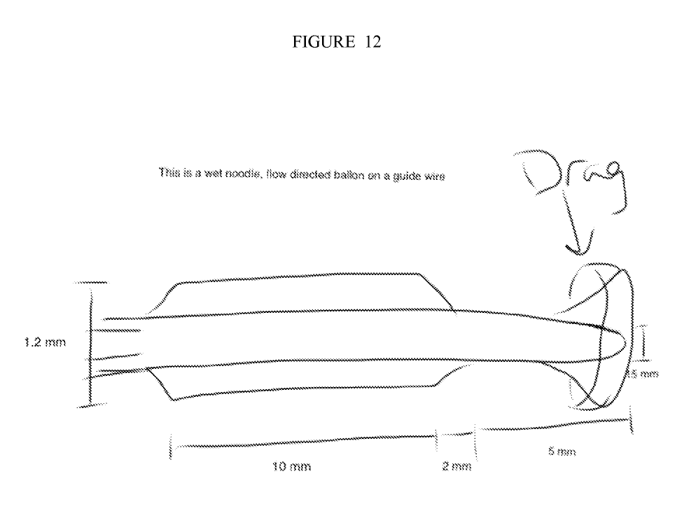

FIG. 12 shows a device that will use the vascular flow to aid in locating and positioning within the OA. There are several features that are detailed here, but all share a common design element in that they are specifically designed for the OA anatomy and will work with the vascular flow to aid in placement and positioning within the anatomy. An additional design provision may include the ability to work with the IOP device (as detailed in section I). In this use, the flow directed element would take into account the reversal of vascular flow and would follow that flow accordingly, which would aid in the removal of the device from the target anatomy. This feature would simplify the removal of the instrument by reducing the amount of force required to withdraw the device. There are several examples of this design as noted by FIG. 12.

II. Interventional Device—Flow Directed 2

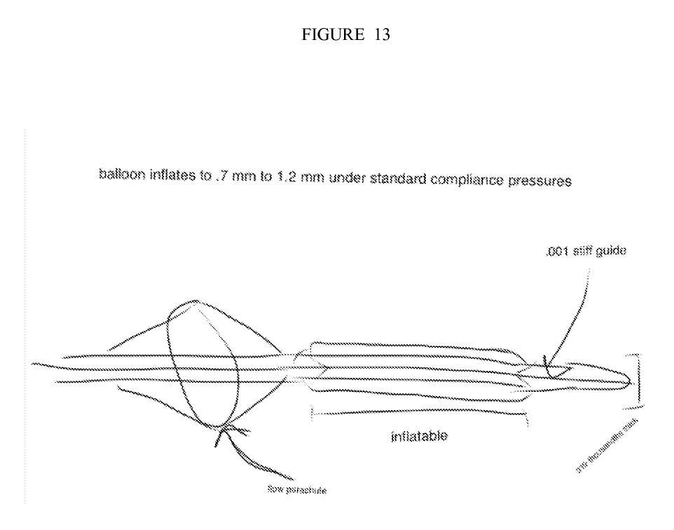

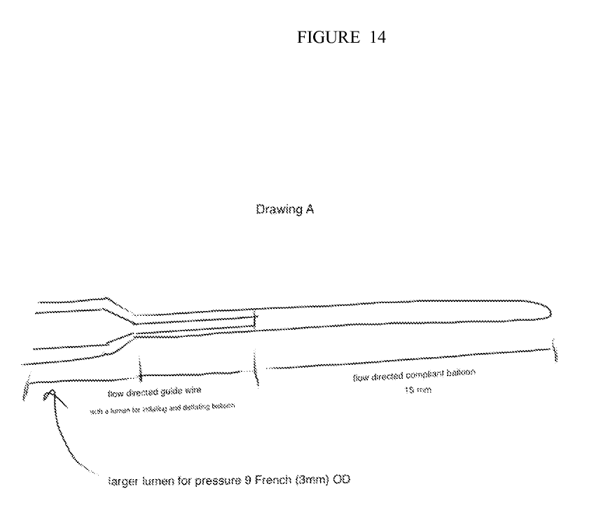

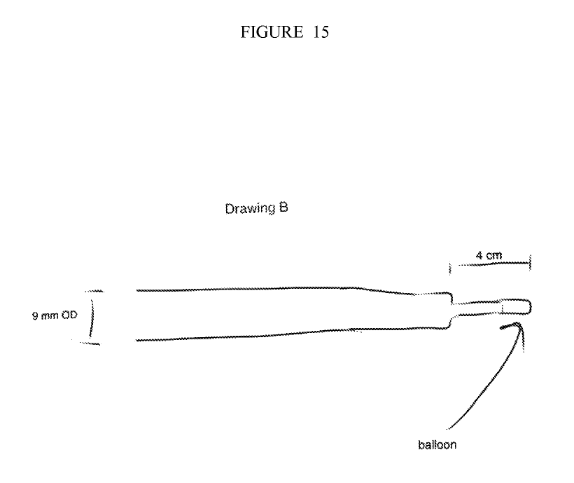

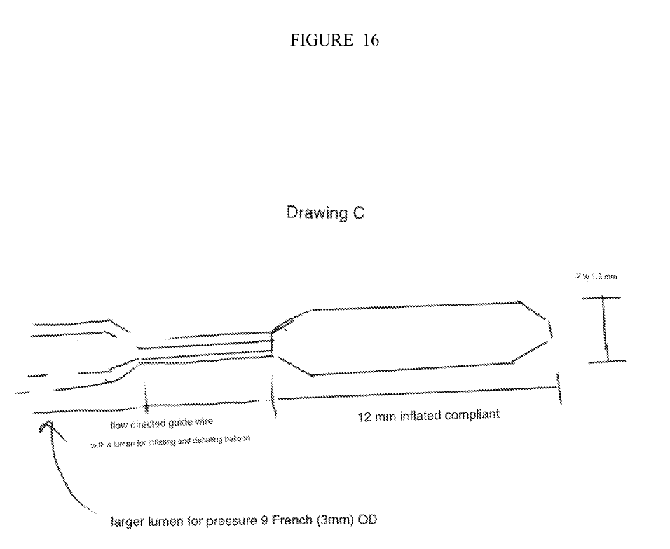

FIGS. 13-16 describe a simple, flow directed balloon that is unified with a large volume delivery catheter. It is a hybrid guidewire/balloon/aspiration device. Novelty is found in the fact that the flow directed balloon and guidewire are a single unit and that the inner diameter of the lumen starts out at around 7-8 French and narrows dramatically for the last 3-4 cm. Delivery of the device into the ostium of the ophthalmic artery may be done by a catheter, that has a 90 degree port at the ostium of the OA. This allows for the very small diameter (0.19 mm OD or smaller) balloon guidewire to enter into the ophthalmic artery and to be pulled into the artery and across the lesion by normal blood flow. The larger diameter of the inner diameter catheter allows for good pressure to be maintained proximal to the balloon facilitating the delivery of contrast agents in addition to saline for balloon inflation.

IJ. IOP Device—General Description

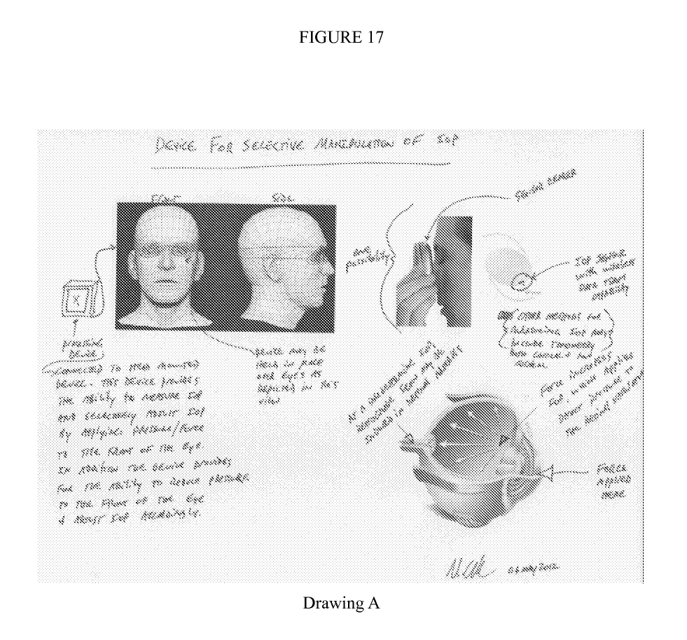

FIG. 17 depicts the general invention behind the device used to manipulate intra ocular pressures (IOP). One element will fit the patients eye(s) such that it may be used to apply pressure to the front of the eye. The eye portion may be held in position with a strap, adhesive, external member or other method that sufficiently accomplishes the task of keeping the eye portion in proper contact with the patient's eye(s). The eye contact portion of the device may be designed to cover and manipulate a single eye, both eyes, one at a time, two at the same time or any combination. Pressure manipulation of the front of the eye will be accomplished by applying a specified amount of direct pressure to the front (typically corneal) area of the eye. This may be accomplished in a variety of ways, including use of pneumatic, hydraulic, gravity and/or other mechanical means of manipulating force over an area. There may be need to combine these forces in such away as to optimize the pressure manipulation. Use of mechanical force manipulation will provide the best methodology and control for removal of force such that the IOP returns to the normal steady state post procedure in a repeatable, desirable manner. A second element will provide for the IOP measurement of the eye under manipulation. There may be several ways to accomplish IOP measurement. These include remote implantable sensor with wired or wireless data transmission capability, corneal tonometry, non-corneal tonometry and/or transpalpebral tonometry. In addition, there may be other ways to accomplish IOP measurements such that pressure values are obtained from the subject eye. A third element will provide the user (physician) with the ability to select pressure and time for the device to interact with the eye in the form of an external control feature. This external control feature may be in the form of a computer, tablet, smart phone or other device that provides the user with the necessary control and feedback information needed to perform the IOP manipulation. This control feature will also contain a feedback loop which will continuously monitor IOP so that a constant pressure may be maintained. This control mechanism will also allow for ramp up/ramp down of pressure, non-constant pressure, time manipulation and/or any combination thereof. This capability will likely be software driven and will provide the user with the ability to custom tailor an IOP manipulation profile for a specific patient. We think that the rate or pressure induced, time at pressure and rate of pressure reduction will be important to the success of the procedure and will design the control mechanism to provide this capability. In addition, the ability to capture, chart and store patient centric data will be an element of this control mechanism.

Use of the device elements as detailed above will allow for the physician to induce retrograde vascular flow (for up to 3 minutes at a time) such that when the interventional device is used, the risk for retinal vasculature embolism is reduced. It is known that at a minimum retrograde flow in the central retinal artery can be maintained for antithrombotic protection and possibly the ciliary arteries. With pressure put on the front of the eye, the blood volume of the choroid layers can be forced back, through the central retinal artery, ciliary arteries and possibly lacrimal arteries.

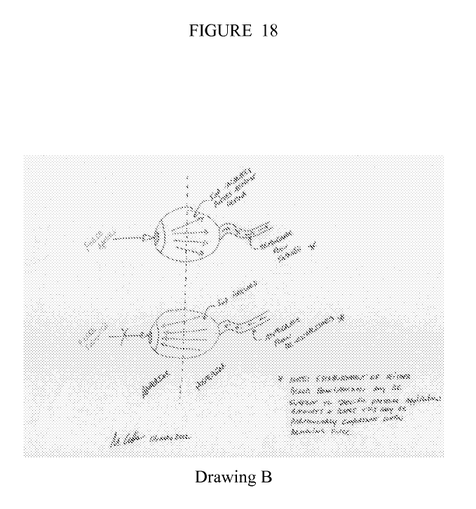

FIG. 18 details how the force applied to the front of the eye will translate into force flowing through the eye, increasing IOP, resulting in retrograde vascular flow. The drawing also details how the removal of force will result in a return to the normal pressure state of the eye. Note the drawing is not anatomically correct.

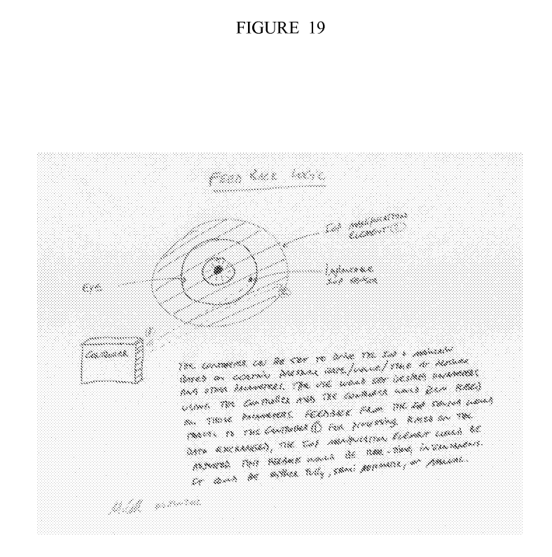

FIG. 19 depicts one embodiment of a feedback loop utilizing an implantable IOP sensor.

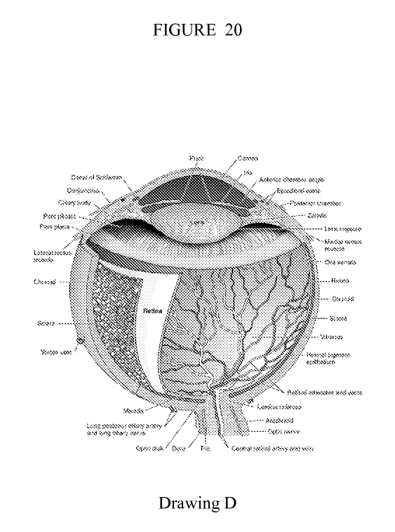

FIG. 20 provides detail associated with the posterior vasculature of the eye. It is for information only.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed as the invention is:

1) An apparatus for treating obstruction of the ophthalmic artery, comprising an IOP device for mechanically applying a force against the front of the eye to increase Intraocular Pressure (IOP) sufficient to temporarily stop antegrade blood flow in the ophthalmic vasculature at the back of the eye and thereby induce retrograde flow in the ophthalmic vasculature; an atherectomy kit for performing an atherectomy upon the ophthalmic artery of a patient in need thereof; and a debris capture device for placement within the ophthalmic vasculature to capture artherectomy debris.

2) A method of treating obstruction of the ophthalmic artery, comprising the steps of: inducing retrograde flow in the ophthalmic vasculature by applying a mechanical force against the front of the eye to increase Intraocular Pressure (IOP) sufficient to temporarily stop antegrade blood flow in the ophthalmic vasculature at the back of the eye; performing an atherectomy upon the ophthalmic artery of a patient in need thereof during retrograde blood flow; and deploying a debris capture device within the ophthalmic vasculature to capture atherectomy debris, wherein the retrograde flow blocks the debris from the atherectomy from flowing downstream and causing an ischemic event.

3) A tissue removal device for treating obstruction of the ophthalmic artery, comprising: a percutaneously delivered tapered corewire ranging in diameter from about 0.19 mm to about 0.88 mm, the corewire disposed within a delivery sheath, said corewire having a tissue cutting element at or near a distal end, said corewire having an integral inflatable balloon section at the distal end as a protective element, and said corewire having an atraumatic tip.

4) A method of use of a shaped tip guidewire, a straight tip guidewire and a guiding catheter, comprising the steps in which the straight tip guidewire is used alone, or in conjunction with the guiding catheter to access the OA from the ICA, once the OA has been cannulated, the shaped tip guidewire is exchanged for the straight tip guidewire for the balance of the procedure.

5) An apparatus for capturing atherectomy debris as it is removed, comprising a single hypo tube cut to contain a combination atherectomy device and distal protection device, wherein the atherectomy device portion fits within a delivery sheath such that the fully expanded diameter is achieved when device is moved out of sheath and into the target anatomy with that fully expanded diameter at 1.4 mm, which compliance to a vessel as small as 0.7 mm., further comprising wherein the atherectomy device portion of the apparatus is expandable, wherein the apparatus has an aspiration device for removal of debris by aspiration via an external sheath.

6) A method for providing treatment for the symptoms related to Wet Age Related Macular Degeneration, comprising the step of removal of stenosis of the OA, thereby restoring normal, or near normal, blood flow to the rear of the eye, including the retina and associated structures.

7) Apparatus that is a single unit, containing a hybrid catheter and flow directed balloon where the OD of single unit being 6-9 french at the thicker, proximal end of the hybrid catheter/balloon with the distal 2-5 CM of the apparatus being 0.12-0.19 mm in diameter of a flow directed guidewire with the final distal portion of the apparatus being 8 to 15 mm of the apparatus being a flow directed balloon, where the flow directed balloon inflates to a maximum of 1.4 mm and a minimum of 0.7 mm, where the balloon is coated with a drug for delivery and compression into the wall of the arterial source with the stenotic lesion.
